(19) United States
(12) Patent Application Publication
(10) Pub. No.: US 2019/0048381 A1
(43) Pub. Date: Feb. 14, 2019

(54) METHODS OF PRODUCING LIPIDS

(71) Applicant: C16 Biosciences, Inc., Somerville, MA (US)

(72) Inventors: Harold M. McNamara, Somerville, MA (US); Shara Ticku, San Antonio, TX (US); David Heller, Cambridge, MA (US); Andrew Shumaker, Somerville, MA (US)

(21) Appl. No.: 16/005,163

(22) Filed: Jun. 11, 2018

Related U.S. Application Data
(60) Provisional application No. 62/517,690, filed on Jun. 9, 2017.

(51) Int. Cl. C12P 39/00; C12P 7/64

(52) U.S. Cl. CPC C12P 39/00; C12P 7/6409

(57) ABSTRACT

Described herein are microoganisms and methods for producing lipids by co-culturing a photosynthetic microorganism with a heterotrophic microorganism to produce a culture medium having a titer of lipids.

### RELATED APPLICATIONS

This application claims the benefit of priority to U.S. Provisional Patent Application Ser. No. 62/517,690 filed Jun. 9, 2017.

### BACKGROUND

Palm oil production is a leading contributor to tropical deforestation, resulting in habitat destruction, increased carbon dioxide emissions, and local smog clouds across South East Asia. Palm oil is widely used for food, as a biofuel precursor, and in soaps and cosmetics. The global demand for palm oil is approximately 57 m tonnes and is steadily increasing.

Alternatively, oleaginous yeast offers a highly credible renewable substitute to conventional palm oil production. Over 80 species of oleaginous yeast are known, many of which have been demonstrated to catabolize a wide range of mono- and oligosaccharides in lignocellulose hydrolyzates. Many of the yeasts have demonstrated a high tolerance to furfurals and organic acid inhibitors and can be cultured at low pH, ideal for the industrial production of oil from lignocellulosic sources such as stover, forestry wastes, or energy crops. While the majority of the yeasts produce predominantly monounsaturated esters, some species are capable of producing oils higher in saturates akin to palm oil.

In order for a microbial oil to offer an economically viable substitute for commodity vegetable oils, they must be produced at high yield from low input costs. Oils produced from single cell cultures (‘single-cell oils’) are in general constrained due to limitations of individual microbes. For example, heterotrophic yeast cultures require sugar input, driving unit costs. Alternatively, phototrophic microbes such as algae (green algae and cyanobacteria) can be grown without sugar input, but achieve low cell densities and product titers.

In contrast, the co-culture of multiple microorganisms represents a new paradigm for achieving high process yield from minimal inputs. We describe here multiple embodiments of a co-culture between a photoautotrophic microorganism and a heterotrophic microorganism.

Important figures of merit for these embodiments include yield (in g oil per liter culture) and chemical properties (measured by relative percentage of different fatty acid components). There is a need in the art for new co-culture techniques which produce a diversity of oil chemical profiles.

Accordingly, there is therefore a need in the art for new microorganism cultures, as well as new methods of culturing microorganisms, to provide a renewable substitute for palm oil production.

### SUMMARY

In certain aspects, provided herein are methods of producing lipids comprising co-culturing a photosynthetic microorganism with a heterotrophic microorganism to produce a culture medium having a titer of lipids.

In certain other aspects, provided herein are method of producing lipids, comprising co-culturing a photosynthetic microorganism with a heterotrophic microorganism, wherein at least 30% of a continuous culture dry cell weight is the heterotrophic microorganism.

In certain other aspects, provided herein are methods of producing a lipid product, comprising co-culturing a photosynthetic microorganism with a heterotrophic microorganism to yield a lipid product having a target fatty acid profile.

In certain other aspects, provided herein are co-cultures comprising a photosynthetic microorganism and a heterotrophic microorganism, wherein the co-culture produces a titer of lipids of at least 300 mg/L.

In still other aspects, provided herein are co-culture comprising a photosynthetic microorganism and a heterotrophic microorganism, wherein the heterotrophic microorganism comprises 30% of continuous culture dry cell weight.

In certain other aspects, provided herein are co-cultures comprising a photosynthetic microorganism with a heterotrophic microorganism, wherein the co-culture produces a lipid product with a tailored fatty acid profile.

In certain other aspects, provided herein is a titer of lipids of at least 300 mg/L, wherein the titer of lipids is produced by co-culturing a photosynthetic microorganism with a heterotrophic microorganism.

In certain other aspects, provided herein is a titer of lipids, wherein the titer of lipids is produced by co-culturing a photosynthetic microorganism with a heterotrophic microorganism, wherein at least 30% of the continuous culture dry cell weight is the heterotrophic microorganism.

In certain other aspects, provided herein is titer of lipids with a tailored fatty acid profile, wherein the titer of lipids is produced by co-culturing a photosynthetic microorganism with a heterotrophic microorganism.

### BRIEF DESCRIPTIONS OF THE FIGURES

FIG. 1 is an overall schematic diagram of microbial co-culture.

FIG. 2A represents a laboratory setup of a photobioreactor culture.

FIG. 2B repreent a 2 L scale co-culture in a photobioreactor (R. toruloides grown) FIG. 3 compares a chromatogram of R. toruloides oil to a chromatogram of natural palm oil.

FIGS. 4A and 4B compare chemical compositions of R. toruloides oil and palm oil as measured by GCMS, with FIG. 4A representing the hemical profile of R. toruloides oil in sucrose-rich media (450 mg/L titer) and FIG. 4B representing the chemical profile of natural palm oil from Thailand.

FIG. 5 shows a chromatogram of oil isolated from R. toruloides grown in sugar-restricted co-culture conditions.

FIG. 6 shows the chemical components of oil isolated from R. toruloides, as measured via GCMS (R. toruloides oil in BG11-co (<1% m/v sucrose) (106 mg/L yield)).

FIG. 7 shows a chromatogram of oil isolated from Y. lipolytica grown in sucrose restricted conditions.

FIG. 8 shows the chemical components of oil isolated from Y. lipolytica grown in sucrose restricted conditions (Y. lipolytica in BG11-Co (<1% sucrose m/v) (155 mg/L oil yield)).

### DETAILED DESCRIPTION

In certain aspects, provided herein are methods of producing lipids comprising co-culturing a photosynthetic microorganism with a heterotrophic microorganism to produce a culture medium having a titer of lipids.

### Definitions

“Photosynthetic microorganism” as used herein refers to a unicellular organism which can directly fix carbon from carbon dioxide in the presence of sunlight via photosynthesis. Equivalent to a photoautotrophic microorganism.

“Heterotrophic microorganism” as used herein refers to a unicellular organism which requires the culture medium to be supplied with a carbon source other than carbon dioxide.

“Cyanobacterium” as used herein refers to a phylum of bacteria which obtain their energy via photosynthesis and can fix carbon from carbon dioxide. Equivalently called cyanophyta, or “blue-green” algae.

“Algae” as used herein refers to a broad category of photosynthetic organisms This includes cyanobacteria (“blue-green algae”) as well as green algae (unicellular algae in the kingdom Plantae) and diatoms.

“Oleaginous” as used herein refers to material which contains a significant component of oils, or which is itself substantial composed of oil.

“Oleaginous yeast” as used herein refers to a collection of yeast species that can accumulate a high proportion of their biomass as lipids (namely greater than 20% of dry cell mass).

“Titer” as used herein refers to both a specific product of a bioprocess, as well as the amount of product produced by the bioprocess. In the present examples, titers refer to both the specific oil produced, as well as the amount and yield of oil produced.

“Yield” as used herein refers to the amount of product produced by a bioprocess culture, here measured in units of density (i.e., mass of product per unit culture volume).

“Chemical composition” as used herein refers to the set of component molecular species in an oil sample, as well the relative proportion that each molecular species contributes to total sample mass.

“Fatty acid profile” as used herein refers to how specific fatty acids contribute to the chemical composition of an oil titer.

“Tailored fatty acid profile” as used herein refers to a fatty acid profile in an oil which has been manipulate towards target properties, either by changing culture conditions, the specicies of heterotrophic yeast, or by genetically modifying the heterotrophic yeast.

“Co-culture” as used herein refers to a bioprocess in which two microorganisms are used to perform separate metabolic conversions in a single culture medium. The co-culture can either be simultaneous in time, or separated in time. In the former, all co-culture partners are in growth phase at the same time. In the latter, metabolic conversion steps may be temporally separated. A co-culture process also includes designs in which metabolic conversions are spatially separated into separate compartments, in which the shared culture medium can be exchanged across a semipermeable membrane.

“Dry cell weight/dry cell mass” as used herein refers to the weight or (/mass) of the microorganism cells when collected and separated from an aqueous culture medium.

“Continuous culture” as used herein refers to a long-term culture (greater than 1 week) in which the organisms are maintained in a particular growth phase (either log or stationary).

“Culture medium” a solid, liquid, or semi-solid designed to support the growth of microorganisms or cells.

“BG11” refers to a liquid culture medium designed to support the growth of cyanobacteria. Also “blue-green 11”.

“BG11-co” referts to a modification of BG-11 medium designed to support co-culture described herein. “Standard” BG11 medium is modified into BG11-co through the addition of salt (NaCl) and 4-(2-hydroxyethyl)-1-piperazineethanesulfonic acid (HEPES) buffer to maintain basic pH.

“Rhodoporidium toruloides” refers to a particular species of oleaginous yeast. Previously called Rhodotorula glutinis or Rhodotorula gracilis. Also abbreviated as R. toruloides. This species includes multiple strains with minor genetic variation.

“Synechococcus elongatus” refers to a species of unicellular cyanobacteria, which includes several related strains (including the strains PCC 7942 and UTEX 2973). Also abbreviated as S. elongates.

“Yarrowia lipolytica” refers to a species of oleaginous yeasts which includes multiple closely related strains. Also abbreviated as Y. lipolytica.

“Transformation” as used herein refers to direct altering of the genetic material of a cell or organism through the uptake of exogenous genetic material. Example methods include electroporation, transformation, or vector-mediated transformation (for example, agrobacterium mediated transformation, or AMT).

“Sugar transporter proteins” as used herein refers to transmembrane proteins which can aid in the transport of sugar across the plasma membrane. One example is the Escherichia coli sucrose-proton symporter CscB, and the Escherichia coli lactose permease LacY.

“Gas chromatography-mass spectrometry” refers to a technique in analytical chemistry by which molecular species in a sample are separated via relative affinities to a stationary phase, and then identified according to their mass spectra measured on a mass spectrometer detector. Abbreviated herein as GCMS.

“Fatty acid methyl esters” as used herein refers to methyl ester derivatives of fatty acids. Oil titers described herein may be extracted for analysis in this format. Abbreviated as FAME.

### Microorganisms

The microorganisms provided herein include cultures of microorganisms and cultures that contain mixtures or consortia of different types of microorganisms. In certain embodiments, the photosynthetic microorganism comprises a cyanobacterium (e.g., from genera Synechococcus and Chlorella) or alga.

Cyanobacteria, also known as blue-green algae, blue-green bacteria, or cyanophyta, are a phylum (or division) of gram-negative bacteria that obtain their energy through photosynthesis. Cyanobacteria use chlorophyll-a as a primary light harvesting pigment and a bluish pigment, phycocyanin, as a secondary light harvesting pigment. However, different species may also exhibit different colors including light gold, yellow, brown, red, emerald green, blue, violet, and blue-black. (Raven et al., Biology of Plants, Fourth Edition, 183-185, (1986)). For example, some species express the phycoerythrin pigment, and exhibit a pink or red color. The light photons captured by pigments help drive the conversion of CO₂ and an electron donor (e.g., H₂O, H₂S) to carbohydrate. The photosynthetic reaction also produces oxygen and the oxidized product of the electron donor. Some types of cyanobacteria express the enzyme nitrogenase, and are capable of fixing nitrogen. Such cyanobacteria can convert atmospheric N₂ to ammonia, nitrate (NO₃), nitrite (NO₂), ammonium, urea and some amino acids.

The cyanobacteria used in the present disclosure are preferably of the genus Synechococcus and/or Chlorella.

Cyanobacteria include unicellular species and species that form colonies; colonies of cyanobacteria may also form filamentous structures, as well as sheets and hollow balls. In some cases, a percentage of the cyanobacteria of the present invention display filamentous structures. For example, about 5%, 10%, 15%, 20%, 25%, 30%, 35%, 40%, 45%, 50%, 55%, 60%, 65%, 70%, 75%, 80%, 85%, 90%, or 95% of the cyanobacteria of the present invention display filamentous structures. The terms “about” and “approximately,” as used herein when referring to a measurable value, mean within 10% of a given value or range.

A percentage of the cyanobacteria in the present invention are live organisms. For example, greater than 5%, 10%, 15%, 20%, 25%, 30%, 35%, 40%, 45%, 50%, 55%, 60%, 65%, 70%, 75%, 80%, 85%, 90%, or 95% of cyanobacteria may be live organisms.

The methods and cultures disclosed herein may comprise cyanobacteria and any known microorganism, however, preferably, the composition comprises cyanobacteria and one or more types of heterotrophic microorganisms. Examples of heterotrophic microorganisms include any known strain of yeast. In certain embodiments, the heterotrophic microorganism comprises a yeast (e.g., an oleaginous yeast).

The methods and cultures disclosed herein may also comprise isolated products secreted by cyanobacteria. Such products may be obtained by any method, including, but not limited to culturing the cyanobacteria for a length of time (as described herein), followed by a means to separate the cyanobacteria from the medium. The separated medium is thereby enriched with secretion products produced by the cyanobacteria. Means for separation may include, but are not limited to: centrifugation, counter-current membrane filtration, filtration, filtration with a pore filter, separation, decanting, a combination of separation and decanting, microfiltration, and/or any combination of the foregoing.

As described herein, in some embodiments, the inventions of the present disclosure encompass a culture (or co-culture) comprising cyanobacteria and at least one heterotrophic microorganism. In some embodiments, the cyanobacteria in the culture may be homogenous. In some embodiments, the cyanobacteria are made up of a heterogenous population of different species. In some embodiments, the heterotrophic microorganisms are homogenous; in other cases, they heterotrophic microorganisms are made up of a heterogenous population of different species. In some embodiments, the culture of the present invention contains cyanobacteria and at least one heterotrophic microorganism. In some embodiments, the culture of the present invention contains at least one, or more than one, species of cyanobacteria and, at least one, or more than one of any heterotrophic microorganism described herein.

In some embodiments, the culture of the present invention contains consortia or groups of cyanobacteria and/or yeast and/or bacteria and/or Archaea. In some embodiments, a consortia includes two or more distinct genera, species, or strains of cyanobacteria and/or yeast (and/or, e.g., bacteria, Archaea) chosen to specifically to, for example, improve resilience (e.g., to predation, competition, etc.), increase sugar production, increase bioproduct production (e.g., ethanol, butanol, etc.), and/or increase overall system productivity. In further embodiments, selected organisms may provide metabolites required by or consumed by other members of the consortia (e.g., fixed nitrogen, etc.). In further embodiments, genera, species, or strains of cyanobacteria and/or yeast chosen for inclusion in cultured consortia or groups are obtained from a specific geographical region or location, or from a set of different geographical regions. Different geographical locations or regions may be positioned at different latitudinal gradients, for example. The geographical locations or regions may be located in a certain continent (e.g., North America, South America, Antarctica, Africa, Europe, Asia, or Australia, or, in a certain country (e.g., United States, etc.). The geographical locations or regions may be located in a specific area of a continent or country (e.g., western, eastern, northern, southern, south-western, south-eastern, north-western, northeastern, etc.). In some embodiments, genera, species, or strains of cyanobacteria and/or yeast chosen for inclusion in cultured consortia or groups are subjected to one or more of the natural selection and/or directed evolution processes described herein.

In some embodiments, the yeast may be in haploid or diploid forms. The yeasts may be capable of undergoing fermentation under anaerobic conditions, aerobic conditions, or both anaerobic and aerobic conditions.

The ability of yeasts to use different sugars can differ depending on whether they are grown aerobically or anaerobically. All strains of yeast may use ammonia and urea as the sole nitrogen source, but cannot utilize nitrate since they lack the ability to reduce it to ammonium. Yeast, in general, can use most amino acids, small peptides and nitrogen bases as a nitrogen source. Amino acids of potential use as a nitrogen source for yeast include but are not limited to: alanine, arginine, aspartic acid, asparagine, glutamic acid, glutamine, methionine, isoleucine, leucine, phenylalanine, proline, serine, threonine, tryptophan, tyrosine, and valine. Histidine, glycine, cystine and lysine are, however, not readily utilized.

Yeasts also generally may have a requirement for phosphorus, which is assimilated as a dihydrogen phosphate ion, and sulfur, which can be assimilated as a sulfate ion or as organic sulfur compounds like the amino acids methionine and cysteine. Some metals such as magnesium, iron, calcium, and zinc also may enhance yeast growth.

This disclosure also includes any species yeast of the genus Rhodosporidium, Yarrowia, Metschnikowia, and Candida, including, but not limited to: Rhodosporidium toruloides, Yarrowia lipolytica, and Metschnikowia pulcherrima.

A percentage of the heterotrophic microorganisms in the present invention are live organisms. For example, greater than 5%, 10%, 15%, 20%, 25%, 30%, 35%, 40%, 45%, 50%, 55%, 60%, 65%, 70%, 75%, 80%, 85%, 90%, or 95% of the heterotrophic microorganisms may be live organisms.

The microorganisms of the present disclosure often exist in nature originally. For example, cyanobacteria may be present in sea water at a concentration of greater than 1, 2, 5, 10, 10², 10³, 10⁴, 10⁵, 10⁶, 10⁷, 10⁸, 10⁹, 10¹⁰, 10¹¹, 10¹², 10¹³ cells/ml. In some embodiments, the microorganisms of the present disclosure are cultured at a particular concentration. For example, the cyanobacteria and/or heterotrophic microorganisms may be present in a culture at a concentration of greater than 1, 2, 5, 10, 10², 10³, 10⁴, 10⁵, 10⁶, 10⁷, 10⁸, 10⁹, 10¹⁰, 10¹¹, 10¹², 10¹³ cells/ml. In some embodiments, the cyanobacteria and/or heterotrophic microorganisms are cultured for greater than mid-log phase growth. In some embodiments, the cyanobacteria and/or heterotrophic microorganisms are cultured for greater than 1, 5, 10, 15, 20, 25, 30, 40, 45, 50, 55, 60, 70, 75, 80, 90, 100, 150, or 200 days.

The microorganisms of the present disclosure, in some embodiments, are grown from isolates obtained from nature (e.g., wild-types) in geographically-specific sites. In further embodiments, wild-type strains are subjected to natural selection to enhance desired traits (e.g., tolerance of certain environmental conditions such as temperature, salt concentration, pH, oxygen concentration, EtOH concentration, nitrogen concentration, etc.). For example, a wild-type strain (e.g., yeast) is selected for its ability to grow and/or ferment in a culture of a specific salt solution, e.g., 3 to 4% NaCl). In other embodiments, wild-type strains are subjected to directed evolution to enhance desired traits (e.g., sugar production, salt tolerance, bioproduct formation, etc.). In some embodiments, the cultures of microorganisms are obtained from culture collections exhibiting desired traits. In further embodiments, strains selected from culture collections are further subjected to directed evolution and/or natural selection in the laboratory. In some preferred embodiments, cyanobacteria (or other heterotrophic organisms) are subjected to directed evolution and selection for a specific property (e.g., rate of sugar production), while yeast are subjected to natural selection to enhance a specific a property (e.g., salt tolerance). The natural selection of yeast may be for any number of properties, including, but not limited to: growth rate at a particular salinity, fermentation production at a particular salinity. In some embodiments, the cyanobacteria (or other microorganism) and/or yeast (or other heterotrophic microorganism) is selected for its ability to survive a range of salinities. In some cases, the cyanobacteria of the present disclosure are able to survive in medium with a relatively low salinity (e.g., about or equal to 0.3%, 0.4%, 0.5%, 0.6%, 0.7%, 0.8%. 0.9%, 1.0% salt concentration).

In light of the disclosure provided herein, those of skill in the art will recognize that directed evolution generally involves three steps. The first step is diversification, wherein the population of organisms is diversified by increasing the rate of random mutation creating a large library of gene variants. Mutagenesis can be accomplished by methods known in the art (e.g., chemical, ultraviolet light, etc.). The second step is selection, wherein the library is tested for the presence of mutants (variants) possessing the desired property using a screening method. Screens enable identification and isolation of high-performing mutants. The third step is amplification, wherein the variants identified in the screen are replicated. These three steps constitute a “round” of directed evolution. In some embodiments, the microorganisms of the present disclosure are subjected to a single round of directed evolution. In other embodiments, the microorganisms of the present disclosure are subjected to multiple rounds of directed evolution. In various embodiments, the microorganisms of the present disclosure are subjected to 2, 3, 4, 5, 6, 7, 8, 9, 10, 20, 30, 40, 50, 60, 70, 80, 90, or 100 or more rounds of directed evolution. In each round, the organisms expressing the highest level of the desired trait of the previous round are diversified in the next round to create a new library. This process may be repeated until the desired trait is expressed at the desired level.

The cyanobacteria may be generated by a process of directed evolution. In some cases, a strain of cyanobacteria used in the inventions of the disclosure is generated from directed evolution to select for a strain that tolerates a certain environment condition (e.g., temperature, salt concentration, pH, oxygen concentration, EtOH concentration, nitrogen concentration, etc.). In some cases, a strain of cyanobacteria used in the inventions of the disclosure is generated from directed evolution to select for a strain that tolerates higher concentrations of ethanol. An approach to selecting such a strain is to grow cyanobacteria cultures in increasing levels of ethanol, and then to amplify surviving organisms.

The methods and compositions described herein (e.g., the methods of directed evolution) may increase the rate of sugar production by cyanobacteria, or other organisms. Often, the increase in rate of sugar production is by more than 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 50, 75, or 100-fold over the original rate (e.g., rate of the wild-type organism or rate of the starting organism) of the sugar production for the cyanobacteria, or other organism. Similarly, the methods and compositions described herein may increase the total quantity of sugar production by cyanobacteria or other organisms, over a period of time. For example, the increase in quantity of sugar produced may be greater than 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 50, 75, or 100-fold over a specified time period, when compared to the quantity of sugar produced by the original (e.g., wild-type organism, starting organism) over the same time period.

In some examples, cyanobacteria, which normally secrete sugars at the rate of approximately 2% (w/v) per 24 hours (e.g., 20 grams sugar per liter of solution per day), are made to secrete sugars at an increased rate. The cyanobacteria are subjected to conditions to enable the cyanobacteria to produce sufficient quantities of sugar to support yeast growth, fermentation, and ethanol production by application of the techniques and processes described herein. In further cases, cyanobacteria subjected to the techniques and processes described herein secrete sugars at the rate of 2%, 4%, 6%, 8%, 10%, 12%, 14%, 16%, 18%, or 20% (w/v), or more, per 24 hours. In further examples, sugar producing cyanobacteria cultures include 2, 5, 10, 10², 10³, 10⁴, 10⁵, 10⁶, 10⁷, 10⁸, 10⁹, 10¹°, 10¹¹, 10¹², 10¹³ cyanobacteria cells/ml. In still further embodiments, sugar producing cyanobacteria cultures include 10⁴ to 10⁹ cyanobacteria cells/ml.

The cyanobacteria of the present disclosure may be genetically modified, while in other cases, the cyanobacteria of the present disclosure are not genetically modified. Similarly, the heterotrophic microorganisms of the present disclosure may be genetically modified, while in other cases, the heterotrophic microorganisms are not genetically modified. In some cases, the cyanobacteria are a mixture of genetically modified and wild-type microorganisms. In some cases, the heterotrophic microorganisms are a mixture of genetically-modified and wild-type microorganisms. As described further herein, at times the present invention comprises both wild-type cyanobacteria and wild-type heterotrophic microorganisms; wild-type cyanobacteria and genetically-modified heterotrophic microorganisms; genetically-modified cyanobacteria and wild-type heterotrophic microorganisms; or genetically-modified cyanobacteria and genetically-modified heterotrophic microorganisms. Also described further herein, at times the present invention comprises cyanobacteria and/or heterotrophic microorganisms that have been subjected to natural selection and/or directed evolution. In some cases, the cyanobacteria are a mixture of microorganisms modified by natural selection and/or directed evolution and wild-type microorganisms. In some cases, the cyanobacteria are a mixture of microorganisms modified by natural selection and/or directed evolution and microorganisms genetically-modified by other methods disclosed herein. In some cases, the heterotrophic microorganisms are a mixture of microorganisms modified by natural selection and/or directed evolution and wild-type microorganisms. In some cases, the heterotrophic microorganisms are a mixture of microorganisms modified by natural selection and/or directed evolution and microorganisms genetically-modified by other methods disclosed herein.

Genetic Modifications and Other Features of Microorganisms

The cyanobacteria and/or heterotrophic microorganisms can be genetically modified by any method known in the art (e.g., transfection, electroporation, etc.). The genetic modifications may also be of any type known in the art. The genetic modification may be directly or indirectly related to the production of a fermentation product, e.g., ethanol or butanol. The genetic modification may also have no relation to the generation of a fermentation product, or only a very attenuated relationship to the generation of a fermentation product. In some cases, the genetic modification may be directly or indirectly related to the production of a fermentable product, e.g., sugar. The genetic modification may also have no relation to the generation of a fermentable product, or only a very attenuated relationship to the generation of a fermentable product.

In some emodiments, the the cyanobacteria are genetically modified to express sugar transport proteins.

The cyanobacteria and/or heterotrophic microorganisms disclosed herein may be genetically-modified in order to regulate heterotrophic biochemical pathways, express proteins or enzymes, or to increase tolerance to environmental conditions (e.g., temperature, salt concentration, pH, oxygen concentration, EtOH concentration, nitrogen concentration, etc.) during fermentation.

In some embodiments, the cyanobacteria are genetically modified to produce, or improve production of a nutrient (e.g., sugar, nitrogen compound, trace element) that is consumed by the heterotrophic microorganism. For example, if the heterotrophic microorganism metabolizes (or catabolizes) six-carbon sugars, the cyanobacteria may be genetically modified to produce glucose. In some embodiments, the heterotrophic microorganisms are genetically modified to catabolize a nutrient (e.g., sugar, nitrogen compound, trace element) that is secreted by cyanobacteria, or genetically-modified cyanobacteria. For example, if the cyanobacteria secrete a specific nitrogen compound, the heterotrophic microorganism may be genetically engineered to catabolize that specific nitrogen compound.

In some embodiments, the cyanobacteria and/or heterotrophic microorganisms are genetically-modified in any other manner known in the art. For example, they may be genetically modified to express a fluorescent protein, e.g., green fluorescent protein, red fluorescent protein. In some embodiments, they may be genetically modified to tolerate a particular type of environment (e.g., high temperature). In some embodiments, they may be genetically modified to tolerate high-salt concentrations. In some embodiments, they may not be genetically modified to tolerate high-salt concentrations. For example, in some cases the cyanobacteria and/or heterotrophic microorganisms are genetically-modified to express a gene for salt tolerance (e.g., the HAL1 yeast gene). In some embodiments, the cyanobacteria and/or heterotrophic microorganisms are not genetically-modified to express a gene for salt tolerance (e.g., HAL1 yeast gene).

In some embodiments, the modifications to the microorganisms may result in regulation of heterotrophic biochemical pathways, expression of certain genes (e.g., genes encoding saccharolytic enzymes), or increased tolerance to environmental conditions during fermentation. Cyanobacteria or heterotrophic organisms described herein may be transformed with heterologous polynucleotides encoding one or more genes for the pathway, enzyme, or protein of interest. In another embodiment, cyanobacteria or heterotrophic organisms are transformed to produce multiple copies of one or more genes for the pathway, enzyme, or protein of interest. In one embodiment, Cyanobacteria or heterotrophic organisms described herein are transformed with heterologous polynucleotides encoding one or more enzymes for the hydrolysis and/or fermentation of a hexose. As a result, said cyanobacteria or heterotrophic microorganism may have the improved ability to produce ethanol (e.g., improved productivity levels or yields) compared to cyanobacteria or heterotrophic microorganisms that are not transformed.

Microorganism Cultures

In some embodiments, a composition of the invention comprises both microorganisms and a particular culture medium. In some cases, one or more microorganisms is cultured in one type of medium first and then transferred to a different type of medium. For example, one or more heterotrophic organisms described herein may be cultured in a specific medium and then transferred to medium comprising cyanobacteria, or products (or nutrients) secreted by cyanobacteria. Similarly, the cyanobacteria described herein may be cultured in a specific culture medium and then transferred to a different medium. In some embodiments, the medium used for the cyanobacteria/heterotrophic microorganism co-culture is the same medium that was previously used to culture the cyanobacteria and/or the heterotrophic microorganisms in the culture. In some embodiments, the medium used for the cyanobacteria/heterotrophic microorganism co-culture is different from the culture medium previously used to culture the cyanobacteria and/or the heterotrophic microorganisms.

An advantage of some of the embodiments of the current disclosure is that the cyanobacteria need not be grown on defined medium. Instead, the cyanobacteria may be grown on sea water or filtered sea water. In some embodiments, the sea water or filtered sea water is monitored for input seawater parameters (e.g., PO.sub.4, trace elements, salts, temperature, pH, etc.). In further embodiments, growth conditions are managed by specific adjustment of individual parameters falling outside specific ranges. In some embodiments, the sea water or filtered sea water is monitored for one or more organisms of interest (e.g., cyanobacteria, predators, competitors, etc.). In further embodiments, growth conditions are managed by adjustment of the concentration and/or presence of one or more particular organisms of interest. In some embodiments, it may be useful to grow the cyanobacteria for a certain amount of time on defined medium.

In some embodiments, the heterotrophic microorganisms receive a substantial amount of growth media (e.g., nutrients, catabolites) from products released by the cyanobacteria. In some cases, the heterotrophic microorganisms receive at least 1%, 1.5%, 2.0%, 2.5%, 3.0%, 3.5%, 4.0%, 4.5%, 5.0%, 5.5%, 6%, 6.5%, 7%, 7.5%, 8%, 8.5%, 9%, 9.5%, 10%, 15%, 20%, 25%, 30%, 35%, 40%, 45%, 50%, 55%, 60%, 65%, 70%, 75%, 80%, 85%, 90%, 95%, or 100% of their maximum growth requirements for a particular nutrient (e.g., sugar, nitrogen compound) from products released by cyanobacteria.

In some cases, a microorganism co-culture provided herein has a cyanobacteria:heterotrophic microorganism ratio of at least 10⁵:1, 10⁴:1, 10³:1, 100:1, 90:1, 80:1, 85:1, 80:1, 75:1, 50:1, 40:1, 30:1, 20:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:20, 1:30, 1:40, 1:50, 1:75, 1:80, 1:85, 1:90, 1:100, 1:10³, 1:10⁴, or 1:10⁵. In some cases, the foregoing ratio refers to the total cyanobacteria and/or heterotrophic microorganisms in a culture. In some cases, the foregoing ratio refers to a specific strain of cyanobacteria and/or heterotrophic microorganism in a culture.

In some embodiments, the cyanobacteria and/or heterotrophic microorganism cultures (or co-cultures) are cultured for greater than 1, 5, 10, 15, 20, 25, 30, 40, 45, 50, 55, 60, 70, 75, 80, 90, 100, 150, or 200 days. In some embodiments, the cyanobacteria and/or heterotrophic microorganism cultures (or co-cultures) are cultured until they reach greater than mid-log phase growth.

In some embodiments, the cyanobacteria and/or heterotrophic microorganism cultures (or co-cultures) are cultured under a light/dark regime. In some cases, the light/dark regime may be cycles wherein the cultures are cultured under light conditions for a certain amount of hours, followed by culture under dark conditions for a certain amount of hours. For example, a light/dark cycle could be a range of hours of light:hours of dark, such as 8:16, 9:15, 10:14, 11:13, 12:12, 13:11, 14:10, 15:9, or 16:8. Repeated light/dark cycles may be conducted over a period of time, such as a period of time greater than 1, 5, 10, 15, 20, 25, 30, 40, 45, 50, 55, 60, 70, 75, 80, 90, 100, 150, or 200 days. In some cases, the light phase of a light/dark cycle is greater than or equal to 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, or 24 hours. In some cases, the dark phase of a light/dark cycle is greater than or equal to 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, or 24 hours.

In some embodiments, the cyanobacteria and/or heterotrophic microorganism cultures (or co-cultures) are cultured in the presence of solar energy. As used herein, the term “solar energy” includes natural sunlight, and may include direct sunlight and indirect sunlight. In some embodiments, the cyanobacteria and/or heterotrophic microorganism cultures (or co-cultures) are cultured in the presence of artificial light (e.g., 150 W halogen light). In some embodiments, the cyanobacteria and/or heterotrophic microorganism cultures (or co-cultures) are cultured in the absence of solar energy. In some embodiments, the cyanobacteria and/or heterotrophic microorganism cultures (or co-cultures) are cultured in an aerated vessel. In some embodiments, the cyanobacteria and/or heterotrophic microorganism cultures (or co-cultures) are cultured in a non-aerated vessel or under anaerobic conditions. In some embodiments, the cyanobacteria and/or heterotrophic microorganism cultures (or co-cultures) are cultured in a vessel purged of oxygen. In some embodiments, the cyanobacteria and/or heterotrophic microorganism cultures (or co-cultures) are cultured in a vessel purged of nitrogen. In some embodiments, the cyanobacteria and/or heterotrophic microorganism cultures (or co-cultures) are cultured in a culture medium that comprises a fermentation product at a concentration of at least 0.1%, 0.3%, 0.5%, 0.6%, 1.0%, 1.5%, 2.0%, 5.0%, 10%, 20%, 30%, 50%, or 70% (v/v). In some embodiments, the cyanobacteria and/or heterotrophic microorganism cultures (or co-cultures) are able to fix nitrogen. Thus, in some embodiments, the cyanobacteria and/or heterotrophic microorganism cultures (or co-cultures) do not require the addition of fertilizers that comprise nitrogen. In some embodiments, the cultures or co-cultures grow in the absence of exogenous nitrogen, other than atmospheric nitrogen.

In some embodiments, the cyanobacteria and/or heterotrophic microorganism cultures (or co-cultures) are cultured for greater than 1, 5, 10, 15, 20, 25, 30, 40, 45, 50, 55, 60, 70, 75, 80, 90, 100, 150, or 200 days. In some embodiments, the cyanobacteria and/or heterotrophic microorganism cultures (or co-cultures) are cultured until they reach greater than mid-log phase growth.

In some embodiments, prior to being mixed with the heterotrophic microorganism cultures, the cyanobacteria are cultured under a light/dark regime. In some cases, the light/dark regime may be cycles wherein the cultures are cultured under light conditions for a certain amount of hours, followed by culture under dark conditions for a certain amount of hours. For example, a light/dark cycle could be 1a range of hours of light:hours of dark, such as 8:16, 9:15, 10:14, 11:13, 12:12, 13:11, 14:10, 15:9, or 16:8. Repeated light/dark cycles may be conducted over a period of time, such as a period of time greater than 1, 5, 10, 15, 20, 25, 30, 40, 45, 50, 55, 60, 70, 75, 80, 90, 100, 150, or 200 days. In some cases, the light phase of a light/dark cycle is greater than or equal to 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, or 24 hours. In some cases, the dark phase of a light/dark cycle is greater than or equal to 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, or 24 hours.

In some embodiments, prior to being mixed with the heterotrophic microorganism cultures, the cyanobacteria are cultured in the presence of solar energy. In some embodiments, prior to being mixed with the heterotrophic microorganism cultures, the cyanobacteria are cultured in the presence of artificial light (e.g., 150 W halogen light). In some embodiments, prior to being mixed with the heterotrophic microorganism cultures, the cyanobacteria are cultured in the absence of solar energy. In some embodiments, prior to being mixed with the heterotrophic microorganism cultures, the cyanobacteria are cultured in an aerated vessel. In some embodiments, prior to being mixed with the heterotrophic microorganism cultures, the cyanobacteria are cultured in a non-aerated vessel or under anaerobic conditions. In some embodiments, prior to being mixed with the heterotrophic microorganism cultures, the cyanobacteria are cultured in a vessel purged of oxygen. In some embodiments, prior to being mixed with the heterotrophic microorganism cultures, the cyanobacteria are cultured in a vessel purged of nitrogen. In some embodiments, prior to being mixed with the heterotrophic microorganism cultures, the cyanobacteria are cultured in a culture medium that comprises a fermentation product at a concentration of at least 0.01%, 0.02%, 0.03%, 0.04%, 0.05%, 0.06%, 0.07%, 0.08%, 0.09% 0.1%, 0.2%, 0.3%, 0.4%, 0.5%, 0.6%, 0.7%, 0.8%, 0.9%, 1.0%, 1.5%, 2.0%, 5.0%, 10%, 20%, 30%, 50%, or 70% (v/v).

Methods

In certain aspects, provided herein are methods of producing lipids comprising co-culturing a photosynthetic microorganism with a heterotrophic microorganism to produce a culture medium having a titer of lipids. In some embodiments, the titer of lipids is at least 1, 2, 5, 10, 50, 100, 150, 200, 250, 300, 350, 400, 450, 500, 550, 600, 650, 700, 750 mg/L.

In certain aspects, provided herein are method of producing lipids, comprising co-culturing a photosynthetic microorganism with a heterotrophic microorganism, wherein at least 60%, at least 50%, at least 40%, at least 30%, at least 20%, at least 10%, at least 5%, at least 1% of a continuous culture dry cell weight is the heterotrophic microorganism.

In certain other aspects, provided herein are methods of producing a lipid product, comprising co-culturing a photosynthetic microorganism with a heterotrophic microorganism to yield a lipid product having a target fatty acid profile.

In certain embodiments, the lipid product has a fatty acid profile comprising >40% palmitic acid (C16:0). In certain embodiments, the lipid product has a fatty acid profile comprising >40% oleic acid (C18:1). In certain embodiments, the lipid product has a fatty acid profile comprising >10% fatty acids with chain length shorter than 16 carbons. In certain embodiments, the lipid product has a fatty acid profile comprising >10% lauric acid (C12:0). In certain embodiments, the lipid product has a fatty acid profile comprising >75% of combined palmitic and oleic acids.

In certain embodiments, the target fatty acid profile matches crude palm mesocarp oil or a fraction thereof.

In certain embodiments, the lipid product is a titer of lipids of at least 1, 2, 5, 10, 50, 100, 150, 200, 250, 300, 350, 400, 450, 500, 550, 600, 650, 700, 750 mg/L.

In certain embodiments, at least 1, 2, 3, 4, 5, 6, 7, 8, 9, 10, 15, 20, 25, 30, 35, 40, 45, 50, 55, 60% of a continuous culture dry cell weight is the heterotrophic microorganism.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

### EXAMPLES

### Example 1: A Co-Culture of CscB+ PCC 7942 and R. Toruloides

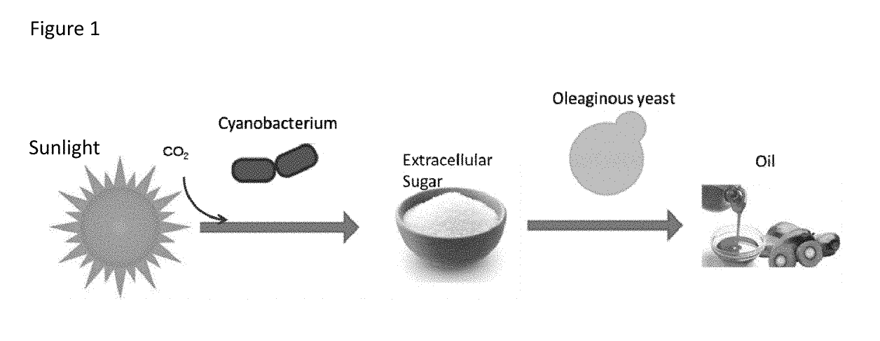

This example consists of a co-culture of the cyanobacterium Synechococcus elongatus PCC 7942 heterologously expressing the proton-sucrose symporter CscB, and the oleaginous yeast Rhodospiridium toruloides. This co-culture subsists in minimal aqueous medium containing trace metals, nitrogen, and salt (bluegreen-11 co-culture medium, ‘BG11-co’). This is an example of the co-culture scheme diagrammed in FIG. 1. A photoautotrophic microorganism and a heterotrophic microorganism are both grown in a basal culture medium based on the cyanobacterial bluegreen-11 medium (BG-11) which contains trace metals and nitrogen. Microorganisms can be grown either in a single compartment (single-pot), or in spatially separated compartments. In the presence of sunlight and carbon dioxide (either from ambient atmospheric gas or from concentrated carbon dioxide sources), the photoautotrophic microorganism fixs carbon in sugar (e.g. sucrose) and then secretes this sugar into the extracellular culture medium. The heterotrophic partner then takes up extracellular sugars and metabolize them into oils.

The cyanobacterium S. elongatus PCC 7942 directs fixed carbon into intracellular sucrose in a saline culture medium. This is an intrinsic osmoprotective response of the strain. When the sucrose-proton symporter CscB is heterologously expressed in PCC 7942, intracellular sucrose is exported into the culture medium in sufficiently basic pH (pH>7.8). In E. coli, CscB imports sucrose when the extracellular solution is acidic relative to the intracellular environment. When the direction of the pH gradient is reversed, the direction of the sucrose transport is similarly reversed. Accordingly, the rate of sucrose production and export is dependent on the culture pH and salt levels. By mapping the sucrose export as a function of medium conditions, metabolic conversion of carbon from gaseous carbon dioxide to oil in the co-culture format can be optimized.

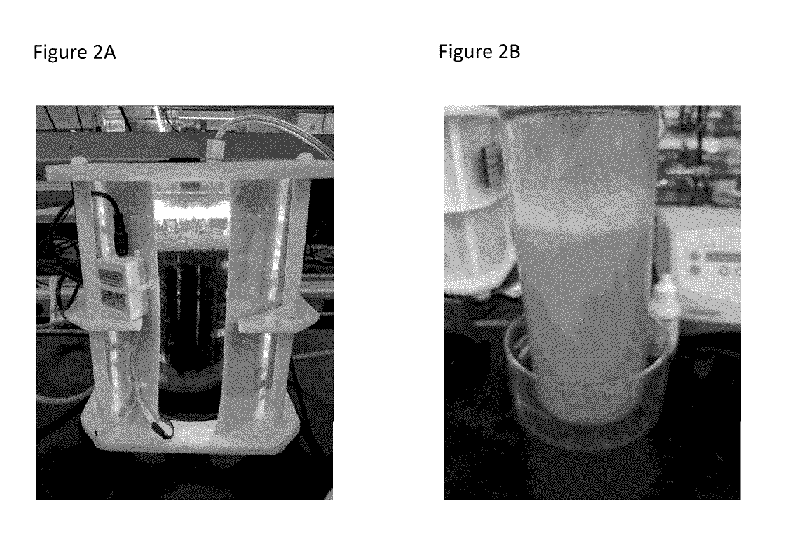

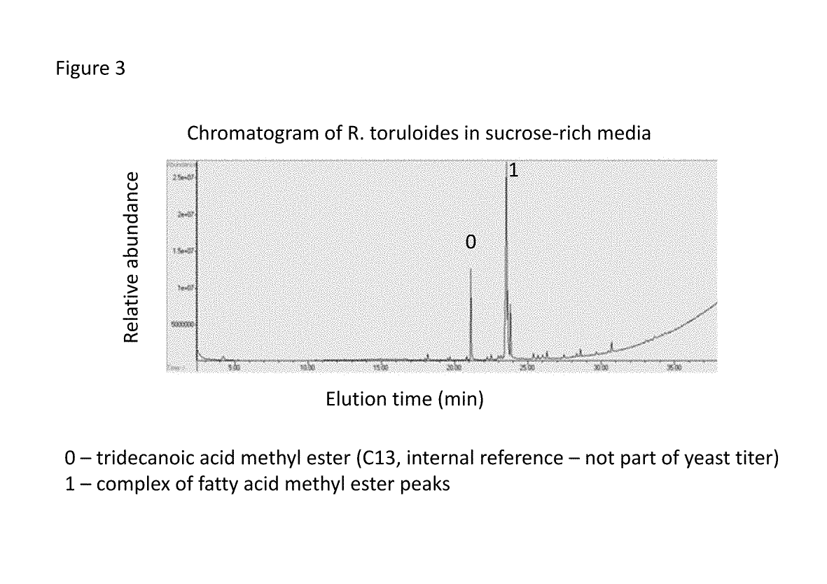

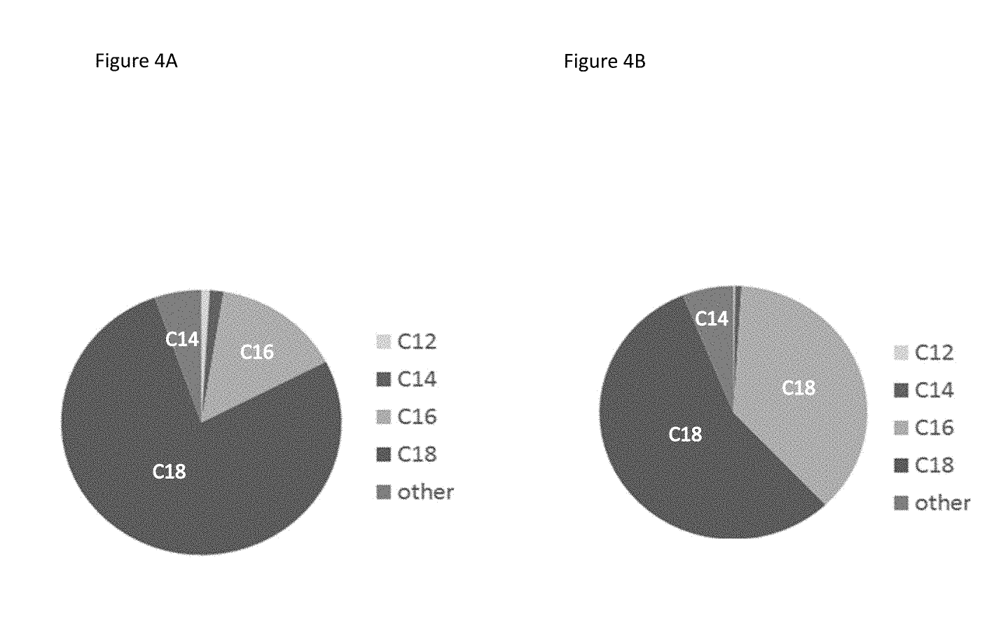

The oleaginous yeast R. toruloides can subsist and proliferate in BG11-co when a sufficient amount of sugar is present in the co-culture medium. FIG. 2 diagrams laboratory realizations of this co-culture in 2 liter photobioreactors. FIGS. 3 and 4 describes how, when R. toruloides is grown in sufficiently rich media conditions, it produces a fatty acid titer which closey matches the lipid profile of crude palm FIG. 4 shows the chemical composition of oil isolated from this sample. Notably, it is rich in the 16- and 18-carbonfatty acids, as is crude palm oil (co-plotted for reference).

### Example 2: A Co-Culture of CscB+ PCC 7942 and R. Toruloides with a Lipid Profile Manipulated by Culture Conditions

Lipid metabolism in oleaginous yeast is known to vary depend on on physical conditions (temperature, pH, salinity) and nutrient availability (sucrose level, C/N ratio). By manipulating the precise conditions of co-culture, the yield and chemical properties of the resultant oil titer can be manipulated and shifted.

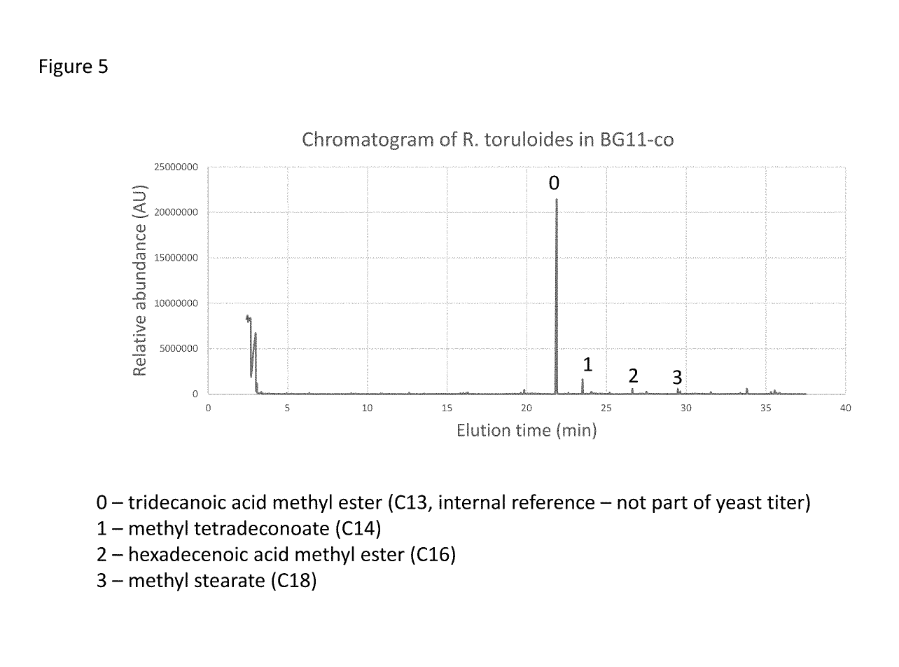

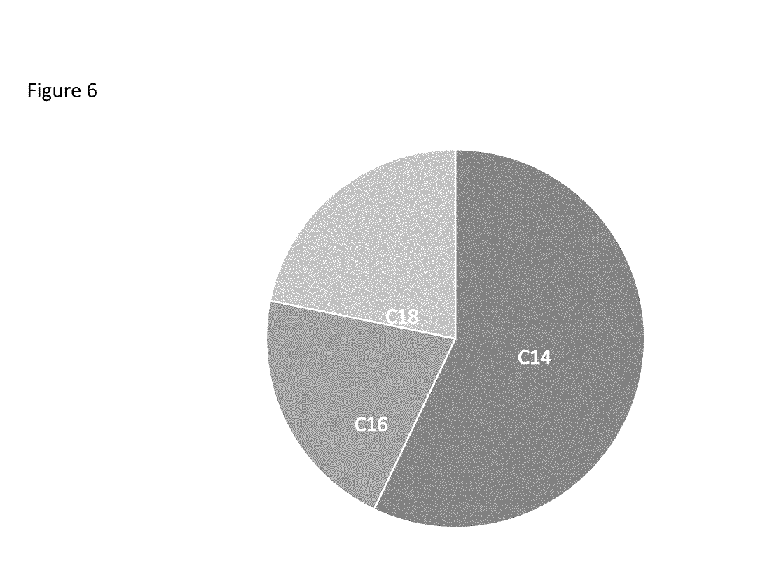

A co-culture of R toruloides in nutrient restricted conditions was performed (<1% sucrose mass/volume). FIG. 5 depicts a chromatogram of the resultant oil titer, and FIG. 6 shows the chemical composition of this titer as analyzed by GCMS after extraction as fatty acid methyl esters. Notably the chemical profile of the R. toruloides oil titer depends strongly on culture conditions. Whereas in rich media 16- and 18-carbon chain lengths dominate, 14-carbon chain lengths are most prominent in this sample. The physical conditions thus constitute a means to manipulate the oil titer produced.

### Example 3: A Co-Culture of CscB+ PCC 7942 and Y. lipolytica

This example consists of a co-culture of the cyanobacterium S. elongatus PCC 7942 heterologously expressing the proton-sucrose symporter CscB, and the oleaginous yeast Yarrowia lipolytica. This co-culture subsists in minimal aqueous medium containing trace metals, nitrogen, and salt (bluegreen-11 co-culture medium, ‘BG11-co’). This is an example of the co-culture scheme diagrammed in FIG. 1. In this example, the photoautotrophic partner functions similarly as in example 1.

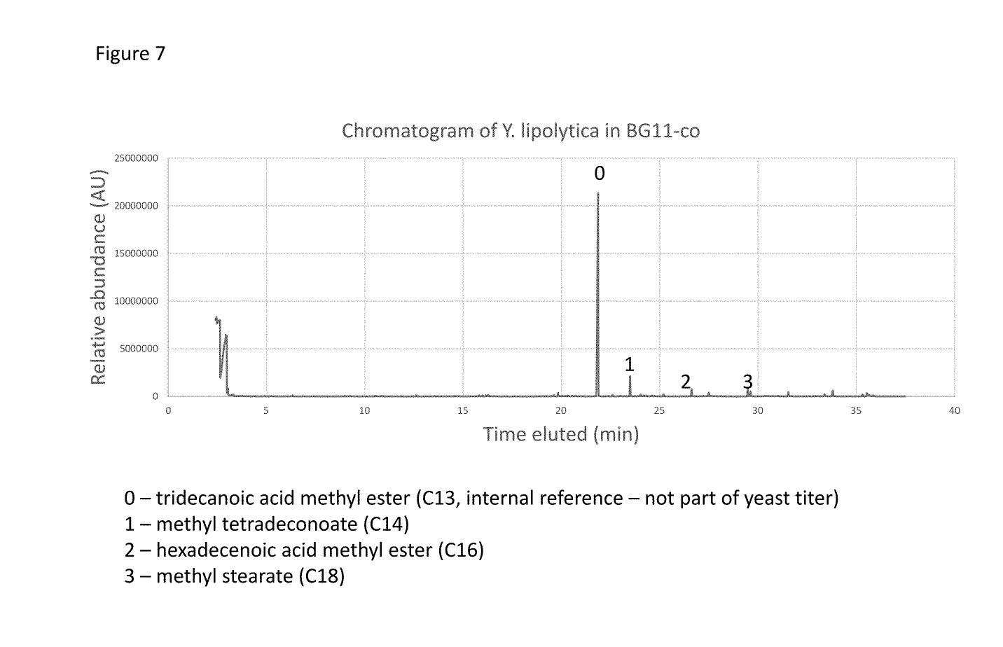

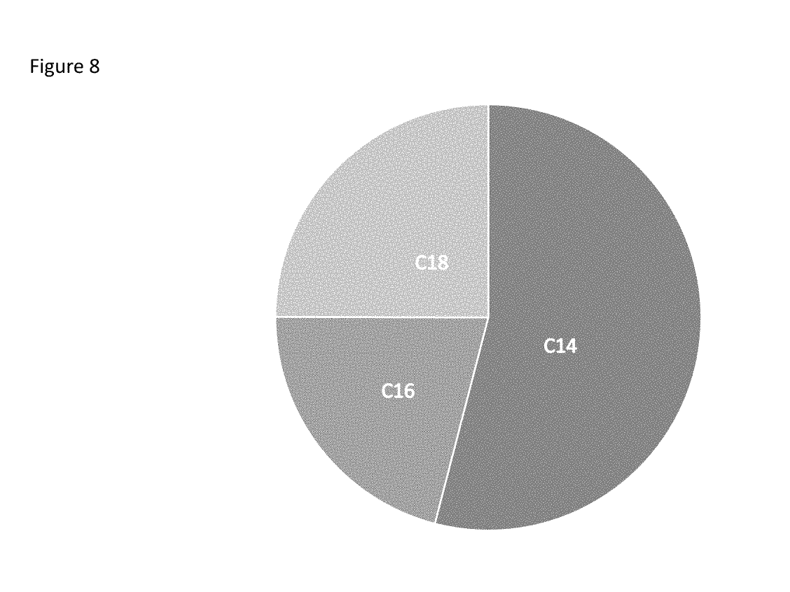

In this example, a different oleaginous yeast was substituted as the heterotrophic partner. Y. lipolytica is evolutionarily distant from R. toruloides and has accordingly distinct lipid metabolism (both in terms of oil yield, in chain length distribution of fatty acid components, and in saturation levels). By substituting different oleaginous yeasts further modification of the microbial oils produced via co-culture can be obtained. Moreover, the use of different yeast species may yield other advantages, which depend on the specific application in question. FIGS. 7 and 8 demonstrate that Y. lipolytica can be deployed in this format, and describe analysis of the resultant oil titer.

### Example 4: a co-culture of CscB+ PCC 7942 and M. pulcherrima

In example 3 described how the heterotrophic co-culture partner could be exchanged for a different oleaginous yeast to produce oils with different titer yields and chemical compositions. This principle generalizes beyond the specific examples of R. toruloides and Y. lipolytica to other oleaginous yeasts. One such example is the oleaginous yeast Metschnikowia pulcherrima, which has recently been demonstrated to accumulate in nutrient-limited growth conditions. By growing photoautotrophic cultures of M. pulcherrima, one can access new fatty acid titers and lipid compositions in accordance with the unique metabolism of this yeast.

1. A method of producing lipids, the method comprising co-culturing a photosynthetic microorganism with a heterotrophic microorganism to produce a culture medium having a titer of lipids of at least 300 mg/L.

2. The method of claim 1, wherein the photosynthetic microorganism comprises a cyanobacterium or alga.

3. (canceled)

4. The method of claim 2, wherein the cyanobacterium is selected from genera Synechococcus and Chlorella.

5. The method of claim 1, wherein the photosynthetic microorganism expresses sugar transport proteins.

6. The method of claim 1, wherein the heterotrophic microorganism comprises a yeast, or an oleaginous yeast.

7. (canceled)

8. The method of claim 6, wherein the yeast is selected from genera Rhodosporidium, Yarrowia, and Candida, or from Rhodosporidium toruloides and Yarrowia lipolytica.

9. (canceled)

10. A method of producing lipids, comprising co-culturing a photosynthetic microorganism with a heterotrophic microorganism, wherein at least 30% of a continuous culture dry cell weight is the heterotrophic microorganism.

11. The method of claim 10, wherein the photosynthetic microorganism comprises a cyanobacterium or alga.

12. (canceled)

13. The method of claim 10, wherein the cyanobacterium is selected from genera Synechococcus and Chlorella.

14. The method of claim 1, wherein the photosynthetic microorganism expresses sugar transport proteins.

15. The method of claim 10, wherein the heterotrophic microorganism comprises a yeast, or oleaginous yeast.

16. (canceled)

17. The method of claim 15, wherein the yeast is selected from genera Rhodosporidium, Yarrowia, Metschnikowia, and Candida.

18. The method of claim 15, wherein the yeast is selected from Rhodosporidium toruloides, Yarrowia lipolytica, and Metschnikowia pulcherrima

19. A method of producing a lipid product, comprising co-culturing a photosynthetic microorganism with a heterotrophic microorganism to yield a lipid product having a target fatty acid profile.

20. The method of claim 19, wherein the lipid product has a fatty acid profile comprising >40% palmitic acid (C16:0).

21. The method of claim 19, wherein the lipid product has a fatty acid profile comprising >40% oleic acid (C18:1).

22. (canceled)

23. The method of claim 19, wherein the lipid product has a fatty acid profile comprising >75% of combined palmitic and oleic acids.

24. The method of claim 19, wherein the target fatty acid profile matches crude palm mesocarp oil or a fraction thereof.

25. The method of claim 19, wherein the photosynthetic microorganism comprises a cyanobacterium or alga.

26. (canceled)

27. The method of claim 26, wherein the cyanobacterium is selected from genera Synechococcus and Chlorella.

28. The method of claim 19, wherein the photosynthetic microorganism expresses sugar transport proteins.

29. The method of claim 19, wherein the heterotrophic microorganism comprises a yeast or an oleaginous yeast.

30. (canceled)

31. The method of claim 30, wherein the yeast is selected from genera Rhodosporidium, Yarrowia, and Candida.

32. The method of claim 30, wherein the yeast is selected from Rhodosporidium toruloides and Yarrowia lipolytica.

33. The method of claim 19, wherein the lipid product is a titer of lipids of at least 300 mg/L.

34. The method of claim 19, wherein at least 30% of a continuous culture dry cell weight is the heterotrophic microorganism.

35.-66. (canceled)

67. A titer of lipids of at least 300 mg/L, wherein the titer of lipids is produced by co-culturing a photosynthetic microorganism with a heterotrophic microorganism.

68. The titer of claim 67, wherein the photosynthetic microorganism comprises a cyanobacterium or alga.

69. (canceled)

70. The titer of claim 69, wherein the cyanobacterium is selected from genera Synechococcus and Chlorella.

71. The titer of claim 67, wherein the photosynthetic microorganism expresses sugar transport proteins.

72. The titer of claim 67, wherein the heterotrophic microorganism comprises a yeast.

73. (canceled)

74. The titer of claim 73, wherein the yeast is selected from genera Rhodosporidium, Yarrowia, and Candida, or from Rhodosporidium toruloides and Yarrowia lipolytica.

75. (canceled)

76. A titer of lipids, wherein the titer of lipids is produced by co-culturing a photosynthetic microorganism with a heterotrophic microorganism, wherein at least 30% of the continuous culture dry cell weight is the heterotrophic microorganism.

77. The titer of claim 76, wherein the photosynthetic microorganism comprises a cyanobacterium or alga.

78. (canceled)

79. The titer of claim 78, wherein the cyanobacterium is selected from genera Synechococcus and Chlorella.

80. The titer of claim 76, wherein the photosynthetic microorganism expresses sugar transport proteins.

81. The titer of claim 76, wherein the heterotrophic microorganism comprises a yeast.

82. (canceled)

83. The titer of claim 81, wherein the yeast is selected from genera Rhodosporidium, Yarrowia, and Candida, or from Rhodosporidium toruloides and Yarrowia lipolytica.

84. (canceled)

85. A titer of lipids with a tailored fatty acid profile, wherein the titer of lipids is produced by co-culturing a photosynthetic microorganism with a heterotrophic.

86. The titer of claim 85, wherein the photosynthetic microorganism comprises a cyanobacterium or alga.

87. (canceled)

88. The titer of claim 85, wherein the cyanobacterium is selected from genera Synechococcus and Chlorella.

89. The titer of claim 85, wherein the photosynthetic microorganism expresses sugar transport proteins.

90. The titer of claim 85, wherein the heterotrophic microorganism comprises a yeast.

91. (canceled)

92. The titer of claim 90, wherein the yeast is selected from genera Rhodosporidium, Yarrowia, and Candida, or from Rhodosporidium toruloides and Yarrowia lipolytica.

93. (canceled)
